(19) United States
(12) Patent Application Publication
(10) Pub. No.: US 2009/0281086 A1
(43) Pub. Date: Nov. 12, 2009

(54) 2 -OXYHETEROARYLAMIDE DERIVATIVES AS PARP INHIBITORS

(75) Inventors: Muhammad Hashim Javaid, Cambridge (GB); Xiao-Ling Fan Cockcroft, Cambridge (GB); Keith Allan Menear, Cambridge (GB); Niall Morrison Barr Martin, Cambridge (GB)

Correspondence Address: MICHAEL BEST & FRIEDRICH LLP, ONE SOUTH PINCKNEY STREET, P O BOX 1806, MADISON, WI, 53701, US

(73) Assignee: KUDOS PHARMACEUTICALS LIMITED, London (GB)

(21) Appl. No.: 12/304,835

(22) PCT Filed: Jun. 15, 2007
(86) PCT No.: PCT/GB2007/002229

§ 371 Date: Dec. 15, 2008

Related U.S. Application Data
(60) Provisional application No. 60/804,851, filed on Jun. 15, 2006.

(51) Int. Cl. A61K 31/496; C07D 409/12; C07D 409/14; A61K 31/498; A61K 31/551; A61P 7/00; A61P 29/00; A61P 9/00; A61P 25/00; A61P 35/00

(52) U.S. Cl. 514/218; 544/379; 544/355; 544/364; 540/575; 514/252.13; 514/249; 514/253.11

(57) ABSTRACT

A compound of the formula (I): and pharmaceutically acceptable salts thereof, wherein: HetA is a C₅ arylene group, wherein the two substituent groups are on adjacent ring atoms, and where the group is further optionally substituted by one halo, amino or C₁₋₇ alkoxy group; Y is —CR^{C1}R^{C2}—(CH₂)ₘ—, where m is 0 or 1, R^{C1} is selected from H, CH₃ and CF₃, and R^{C2} is selected from H and CH₃, or R^{C1} and R^{C2} together with the carbon atom to which they are attached form the 1,1-cyclopropylene group formula (A): R^{N1} and R^{N2} are independently selected from H and R, where R is optionally substituted C₁₋₁₀ alkyl, C₃₋₂₀ heterocyclyl and C₅₋₂₀ aryl; or R^{N1} and R^{N2}, together with the nitrogen atom to which they are attached form an optionally substituted 5-7 membered, nitrogen containing, heterocylic ring; HetB is selected from: (i), where Y¹ and Y³ are independently selected from CH and N, Y² is selected from CX and N and X is H, Cl or F; and (ii), (aa), (bb) Q is O or S.

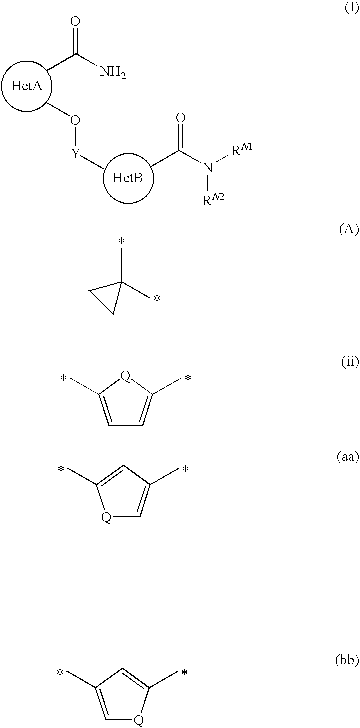

The present invention relates to 2-oxyheteroarylamide derivatives, and their use as pharmaceuticals. In particular, the present invention relates to the use of these compounds to inhibit the activity of the enzyme poly (ADP-ribose) polymerase, also known as poly(ADP-ribose) synthase and poly ADP-ribosyltransferase, and commonly referred to as PARP.

The mammalian enzyme PARP (a 113-kDa multidomain protein) has been implicated in the signalling of DNA damage through its ability to recognize and rapidly bind to DNA single or double strand breaks (D'Amours, et al., Biochem. J., 342, 249-268 (1999)).

Several observations have led to the conclusion that PARP participates in a variety of DNA-related functions including gene amplification, cell division, differentiation, apoptosis, DNA base excision repair and also effects on telomere length and chromosome stability (d'Adda di Fagagna, et al., Nature Gen., 23(1), 76-80 (1999)).

Studies on the mechanism by which PARP modulates DNA repair and other processes has identified its importance in the formation of poly (ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP utilizes NAD to synthesize poly (ADP-ribose) on a variety of nuclear target proteins, including topoisomerase, histones and PARP itself (Rhun, et al., Biochem. Biophys. Res. Commun., 245, 1-10 (1998)).

Poly (ADP-ribosyl)ation has also been associated with malignant transformation. For example, PARP activity is higher in the isolated nuclei of SV40-transformed fibroblasts, while both leukemic cells and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Bioi. Med., 149, 933-938 (1975); and Hirai, et al., Cancer Res., 43, 3441-3446 (1983)).

A number of low-molecular-weight inhibitors of PARP have been used to elucidate the functional role of poly (ADP-ribosyl)ation in DNA repair. In cells treated with alkylating agents, the inhibition of PARP leads to a marked increase in DNA-strand breakage and cell killing (Durkacz, et al., Nature, 283, 593-596 (1980); Berger, N. A., Radiation Research, 101, 4-14 (1985)).

Subsequently, such inhibitors have been shown to enhance the effects of radiation response by suppressing the repair of potentially lethal damage (Ben-Hur, et al., British Journal of Cancer, 49 (Suppl. VI), 34-42 (1984); Schlicker, et al., Int. J. Radiat. Bioi., 75, 91-100 (1999)). PARP inhibitors have been reported to be effective in radio sensitising hypoxic tumour cells (U.S. Pat. No. 5,032,617; U.S. Pat. No. 5,215,738 and U.S. Pat. No. 5,041,653).

Furthermore, PARP knockout (PARP −/−) animals exhibit genomic instability in response to alkylating agents and γ-irradiation (Wang, et al., Genes Dev., 9, 509-520 (1995); Menissier de Murcia, et al., Proc. Natl. Acad. Sci. USA, 94, 7303-7307 (1997)).

A role for PARP has also been demonstrated in certain vascular diseases, septic shock, ischaemic injury and neurotoxicity (Cantoni, et al., Biochim. Biophys. Acta, 1014, 1-7 (1989); Szabo, et al., J. Clin. Invest., 100, 723-735 (1997)). Oxygen radical DNA damage that leads to strand breaks in DNA, which are subsequently recognised by PARP, is a major contributing factor to such disease states as shown by PARP inhibitor studies (Cosi, et al., J. Neurosci. Res., 39, 38-46 (1994); Said, et al., Proc. Natl. Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)).

It has also been demonstrated that efficient retroviral infection of mammalian cells is blocked by the inhibition of PARP activity. Such inhibition of recombinant retroviral vector infections was shown to occur in various different cell types (Gaken, et al., J. Virology, 70(6), 3992-4000 (1996)). Inhibitors of PARP have thus been developed for the use in anti-viral therapies and in cancer treatment (WO 91/18591).

Moreover, PARP inhibition has been speculated to delay the onset of aging characteristics in human fibroblasts (Rattan and Clark, Biochem. Biophys. Res. Comm., 201(2), 665-672 (1994)). This may be related to the role that PARP plays in controlling telomere function (d'Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H. B., et al., Am. J. Physiol. Gastrointest. Liver Physiol., 279, G641-G651 (2000).

Some of the present inventors have previously described (WO 02/36576) a class of 1(2H)-phthalazinone compounds which act as PARP inhibitors. The compounds have the general formula:

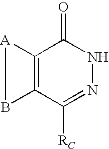

where A and B together represent an optionally substituted, fused aromatic ring and where R_{C} is represented by -L-R_{L}. A large number of examples are of the formula:

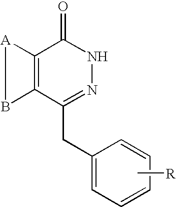

where R represent one or more optional substituents.

In copending applications PCT/GB2005/005017 and U.S. Ser. No. 11/315,528, which are herein incorporated by reference, the following class of compounds has been disclosed as having PARP inhibitory activity:

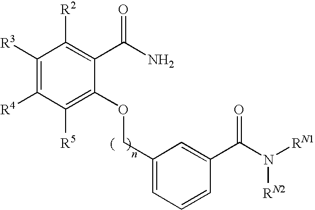

where n is 1 or 2.

The present inventors have now discovered a further class of compounds that inhibit the activity of PARP.

Accordingly, the first aspect of the present invention provides a compound of the formula (I):

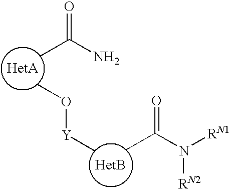

and pharmaceutically acceptable salts thereof, wherein:

HetA is a C₅ arylene group, wherein the two substituent groups are on adjacent ring atoms, and where the group is further optionally substituted by one halo, amino or C₁₋₇ alkoxy group; Y is —CR^{C1}R^{C2}—(CH₂)ₘ—, where m is 0 or 1, R^{C1} is selected from H, CH₃ and CF₃, and R^{C2} is selected from H and CH₃, or R^{C1} and R^{C2} together with the carbon atom to which they are attached form the 1,1-cyclopropylene group:

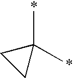

R^{N1} and R^{N2} are independently selected from H and R, where R is optionally substituted C₁₋₁₀ alkyl, C₃₋₂₀ heterocyclyl and C₅₋₂₀ aryl;
or R^{N1} and R^{N2}, together with the nitrogen atom to which they are attached form an optionally substituted 5-7 membered, nitrogen containing, heterocylic ring;
HetB is selected from:

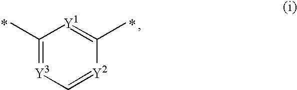

where Y¹ and Y³ are independently selected from CH and N, Y² is selected from CX and N and X is H, Cl or F; and

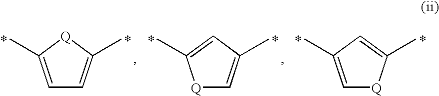

where Q is O or S.

The possibilities for HetB are:

| | Formula | | | |
|---|---|---|---|---|
| | Y¹ | Y² | Y³ | Group |
| 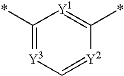 | CH | CH | CH | 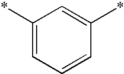 |
| | CH | CX | CH | 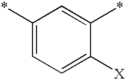 |
| | N | CH | CH | 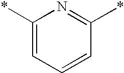 |
| | N | CX | CH | 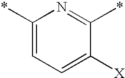 |
| | CH | CH | N | 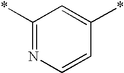 |
| | CH | CX | N | 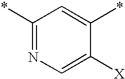 |
| | CH | N | CH | 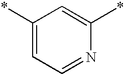 |
| | N | CH | N | 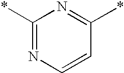 |
| | N | CX | N | 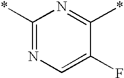 |
| | N | N | CH | 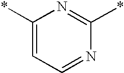 |
| | CH | N | N | 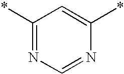 |
| | N | N | N | 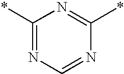 |

| | Q | |
|---|---|---|
| 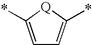 | O | 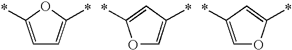 |
| 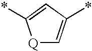 | S | 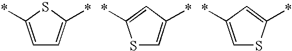 |
| 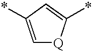 | | |

A second aspect of the present invention provides a pharmaceutical composition comprising a compound of the first aspect and a pharmaceutically acceptable carrier or diluent.

A third aspect of the present invention provides a compound of the first aspect for use in a method of treatment of the human or animal body.

A fourth aspect of the present invention provides the use of a compound as defined in the first aspect of the invention in the preparation of a medicament for:

(a) inhibiting the activity of PARP (PARP-1 and/or PARP-2);
(b) the treatment of: vascular disease; septic shock; ischaemic injury, both cerebral and cardiovascular; reperfusion injury, both cerebral and cardiovascular; neurotoxicity, including acute and chronic treatments for stroke and Parkinsons disease; haemorraghic shock; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery or diseases ameliorated by the inhibition of the activity of PARP;
(c) use as an adjunct in cancer therapy or for potentiating tumour cells for treatment with ionizing radiation or chemotherapeutic agents; and
(d) treating cancer which is deficient in Homologous Recombination (HR) dependent DNA double strand break (DSB) repair activity.

The fourth aspect also provides a compound as defined in the first aspect of the invention for use in the treatment of the conditions detailed above.

In particular, compounds as defined in the first aspect of the invention can be used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the preferred method of administration for the particular agent.

Another further aspect of the invention provides for the use of a compound as defined in the first aspect of the invention in the preparation of a medicament for use as an adjunct in cancer therapy or for potentiating tumour cells for treatment with ionizing radiation or chemotherapeutic agents.

Other further aspects of the invention provide for the treatment of disease ameliorated by the inhibition of PARP, comprising administering to a subject in need of treatment a therapeutically-effective amount of a compound as defined in the first aspect, preferably in the form of a pharmaceutical composition and the treatment of cancer, comprising administering to a subject in need of treatment a therapeutically-effective amount of a compound as defined in the first aspect in combination, preferably in the form of a pharmaceutical composition, simultaneously or sequentially with ionizing radiation or chemotherapeutic agents.

The compounds of the invention may be used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, comprising administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 (NM_{—}002877), RAD51C (NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol. Med., 8(12), 571-6, (2002)). The association of BRCA1 and/or BRCA2 mutations with breast cancer is well-characterised in the art (Radice, P. J., Exp Clin Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some preferred embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The above activity is described WO 2005/053662, which is herein incorporated by reference.

### DEFINITIONS

5-7 membered, nitrogen containing, heterocylic ring: This ring must contain at least one nitrogen atom, and may contain further hetero atoms, i.e. O, S, N.

Examples of five to seven membered nitrogen containing heterocyclic rings are set out below, where Cₙ indicates the number of ring atoms as n.

N₁: pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
N₁O₁S₁: oxathiazine (C₆).

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, C₁₋₁₀ alkyl and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C═CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms.

Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of Cycloalkyl Groups Include, but are not Limited to, Those Derived from

Saturated Monocyclic Hydrocarbon Compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

Unsaturated Monocyclic Hydrocarbon Compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);

Saturated Polycyclic Hydrocarbon Compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

Unsaturated Polycyclic Hydrocarbon Compounds:

camphene (C₁₀), limonene (C₁₀), pinene (C₁₀);

Polycyclic Hydrocarbon Compounds Having an Aromatic Ring:

indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring joined to another ring by a single atom common to both rings.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cyclic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

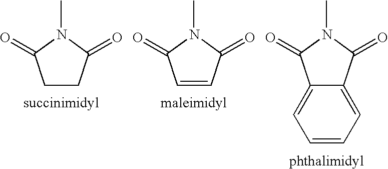

Ureido: —N(R¹)CONR²R³ wherein R² and R³ are independently amino substituents, as defined for amino groups, and R1 is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀ heterocyclyl group, or a C₅₋₂₀ aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

### Further Preferences

The following preferences can apply to each aspect of the present invention, where applicable.

The optional substituent for HetA is preferably selected from the group consisting of C₁₋₇ alkoxy (OMe), Cl and F, and more preferably from F and Cl, with F being the most preferred. In some embodiments, it is preferred that HetA is not substituted.

HetA is preferably a C₅ heteroarylene group containing a single ring heteroatom, e.g. O, S, N.

HetA is preferably derived from thiophene. The sulphur ring atom may be in any position, as shown in the following examples:

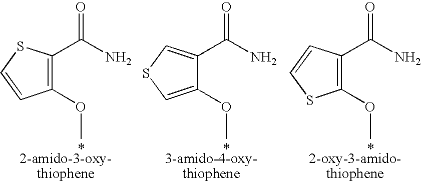

Of these, 2-amido-3-oxy-thiophene is formed from the preferred HetA group, i.e. where the sulphur ring atom is adjacent the amido group.

It is preferred that m is 0.

It is preferred that R^{C2} is H. R^{C1} is also preferably H.

HetB is preferably

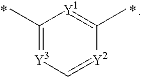

It is preferred that up to two of Y¹, Y² and Y³ are N, and more preferred that one or none of Y¹, Y² and Y³ are N. If one of Y¹, Y² and Y³ are N, it is preferred that this is either Y¹ or Y²

X is preferably selected from H and F, with F being more preferred.

If HetB is

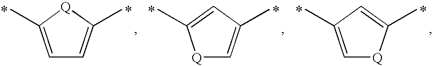

then Q is preferably S. Of these groups,

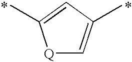

is preferred.

A particularly preferred combination is for HetB to be fluoro-phenylene, R^{C1} and R^{C2} to be H and m to be 0. It is further preferred that HetA and its immediate substituents are:

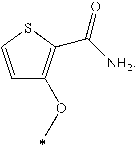

If R^{N1} and R^{N2} are selected from H and R, it is preferred that R^{N1} is H and R^{N2} is R. R is preferably optionally substituted C₁₋₇ alkyl or C₃₋₂₀ heterocylyl, with optionally substituted C₁₋₇ alkyl being more preferred. The C₁₋₇ alkyl group is preferably unsubstituted or substituted with a single substituent, which is preferably selected from a C₅₋₂₀ heterocyclic group (e.g. piperidyl, N-methylpyrrolyl, tetrahydrofuranyl), a C₅₋₂₀ aryl group (e.g. furanyl, phenyl, pyridyl), amino (e.g. dimethyl amino), halo (e.g. Cl, F), hydroxy, ether (e.g. C₁₋₇ alkoxy), thioether (e.g. C₁₋₇ alkylthio). More preferably the single substituent is selected from a C₅₋₂₀ heterocyclic group (e.g. piperidyl, N-methylpyrrolyl, tetrahydrofuranyl), a C₅₋₂₀ aryl group (e.g. furanyl, phenyl, pyridyl), amino (e.g. dimethyl amino), and ether (e.g. C₁₋₇ alkoxy).

When R^{N1} and R^{N2}, together with the nitrogen atom to which they are attached form a 5 to 7 membered, nitrogen containing heterocyclic ring, they preferably form a group of formula II:

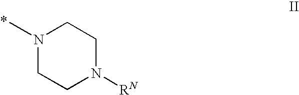

wherein R^{N} is selected from:

### (i) —R^{II};

### (ii) —C(═O)OR^{II};

(iii) —C(═O)NHR^{II};

### (iv) —C(═S)NHR^{II};

### (v) —S(═O)₂R^{II}; and

### (vi) —C(═O)R^{II},

where R^{II} is selected from H, optionally substituted C₁₋₁₀ alkyl, C₃₋₂₀ heterocyclyl and C₅₋₂₀ aryl.

Preferably, R^{N} is selected from:

### (i) —C(═O)NHR^{II};

### (ii) —S(═O)₂R^{II}; and

(iii) —C(═O)R^{II},
where R^{II} is as defined earlier (i.e. H, optionally substituted C₁₋₁₀ alkyl, C₃₋₂₀ heterocyclyl and C₅₋₂₀ aryl).

In the group of formula II, R^{II} is preferably selected from optionally substituted C₁₋₁₀ alkyl and C₅₋₂₀ aryl.

When R^{II} is C₁₋₁₀ alkyl, it is preferably selected from C₁₋₇ alkyl, for example methyl, ethyl, iso-propyl, n-butyl, tert-butyl and C₃₋₆ cycloalkyl, which may be optionally substituted.

When R^{II} is C₁₋₁₀ alkyl, and in particular linear and branched C₁₋₇ alkyl, it may be optionally substituted by one or more, preferably one, groups selected from, for example: C₅₋₂₀ aryl (e.g. phenyl, methyl phenyl, dimethoxy phenyl), C₅₋₂₀ aryloxy (e.g. phenyloxy), C₃₋₂₀ heterocylyl (e.g. piperidinyl), C₁₋₇ alkoxy (e.g. methoxy, benzyloxy).

When R^{II} is C₅₋₂₀ aryl, it is may be selected from optionally substituted C₅₋₆ aryl (e.g. phenyl, oxazole, isoxazole, pyrazole) and optionally substituted C₈₋₁₀ aryl (e.g. benzyloxadiazole, thianopyrazole).

When R^{II} is C₅₋₂₀ aryl, and in particular C₅₋₆ aryl and C₈₋₁₀ aryl, it may be optionally substituted by one or more groups selected from, for example: halo (e.g. F, Cl), C₁₋₇ alkyl (e.g. Me, CF₃), C₅₋₂₀ aryloxy (e.g. phenyloxy), C₁₋₇ alkoxy (e.g. methoxy, benzyloxy), acylamido (e.g. —NH—C(═O)—Me).

When R^{N1} and R^{N2}, together with the nitrogen atom to which they are attached form a 5 to 7 membered, nitrogen containing heterocyclic ring, they may form a group of formula III:

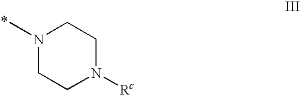

wherein R^{C} is preferably selected from the group consisting of: H; optionally substituted C₁₋₂₀ alkyl; optionally substituted C₅₋₂₀ aryl; optionally substituted C₃₋₂₀ heterocyclyl; optionally substituted acyl, wherein the acyl substituent is preferably selected from C₅₋₂₀ aryl and C₃₋₂₀ heterocylyl (e.g. piperazinyl); optionally substituted amido, wherein the amino groups are preferably selected from H and C₁₋₂₀ alkyl or together with the nitrogen atom, form a C₅₋₂₀ heterocyclic group; and optionally substituted ester groups, wherein the ester substituent is preferably selected from C₁₋₂₀ alkyl groups.

R^{C} is more preferably selected from optionally substituted ester groups, wherein the ester substituent is preferably selected from C₁₋₂₀ alkyl groups.

Where appropriate, the above preferences may be taken in combination with each other.

### Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

### Isomers, Salts, Solvates, Protected Forms, and Prodrugs

Compounds of the invention include the isomers, salts, solvates, protected forms and prodrugs thereof.

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below, as well as its different polymorphic forms.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

### Synthesis

Compounds of the present invention are of formula I:

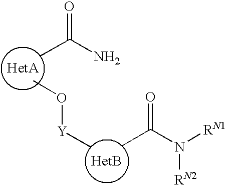

can be synthesised from a compound of formula 2:

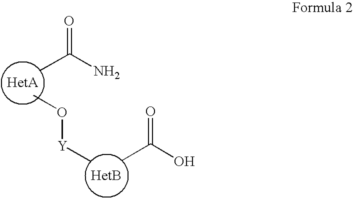

by coupling an amine of formula 3:

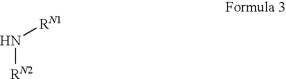

or a precursor or protected form thereof (see below). The coupling may be carried out in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine (Hunig's base), in a solvent, for example dimethylacetamide or dichloromethane, at a temperature in the range of 0° C. to the boiling point of the solvent used.

Alternatively, compounds of the present invention may be synthesised by conversion of a compound of Formula 2 into an activated species, for example an acid chloride or an activated ester such as an N-hydroxysuccinimide ester, using well-known methodologies, and reaction of the activated species with a compound of Formula 3.

Compounds of formula 2 may be obtained by deprotecting compounds of formula 4:

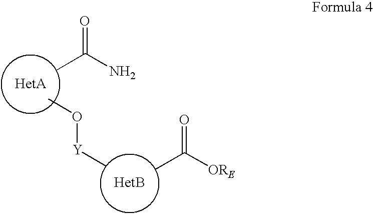

where R^{E} is an optionally substituted, C₁₋₇ alkyl, C₃₋₂₀ heterocyclyl or C₅₋₂₀ aryl group.

Compounds of formula 4 may be synthesised by coupling a compound of formula 5:

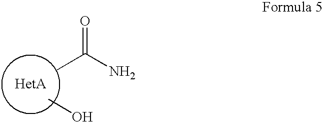

with a compound of formula 6:

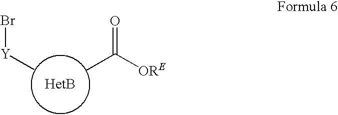

or with a compound of formula 7:

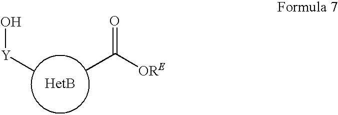

The coupling of compounds of formulae 5 and 6 can be achieved under mildly basic conditions (Williamson reaction), for example, potassium carbonate in acetone.

The coupling of compounds of formulae 5 and 7 can be achieved, using the Mitsunobu reaction (e.g. using diisopropyl azodicarboxylate and triphenylphosphine in acetone)

Compounds of formulae 5, 6 and 7 are either commercially available or readily synthesiable.

When, in compounds of the present invention, R^{N1} and R^{N2} and the nitrogen atom to which they are attached form a group of formula II:

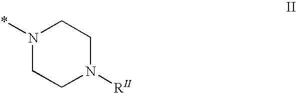

then these compounds can be represented by formula 1a:

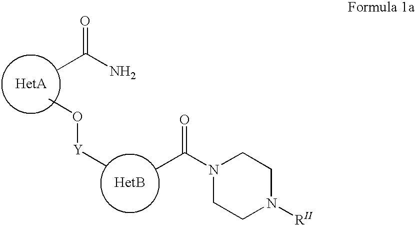

Compounds of formula 1a, wherein R^{II} is H, can be represented by formula 7:

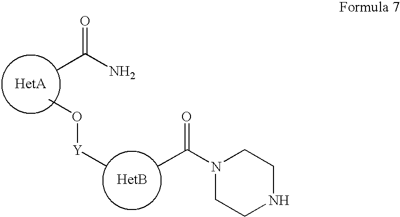

and may be synthesised by deprotection of a protected form of a compound of formula 7, for example a compound of formula 8:

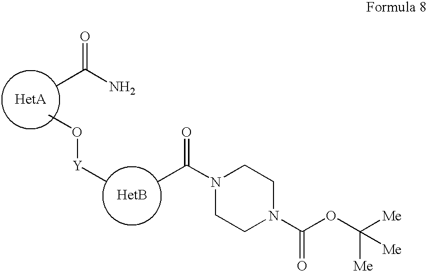

using well known methodologies, for example acid-catalysed cleavage, in the presence of an acid, for example trifluoroacetic acid or hydrochloric acid, in the presence of a solvent, for example dichloromethane or ethanol and/or water, at a temperature in the range of 0° C. to the boiling point of the solvent used.

Compounds of formula 8 may be synthesised from compounds of formula 2 by the previously described methods.

Compounds of formula 1a in which R^{II} is an acyl moiety, can be represented by Formula 9:

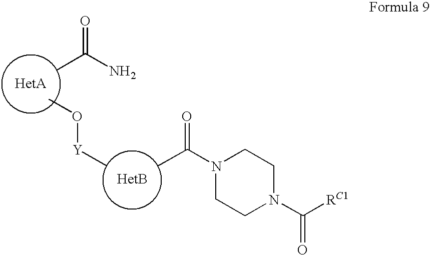

in which R^{C1} is selected from the group consisting of optionally substituted C₁₋₂₀ alkyl, C₅₋₂₀ aryl and C₃₋₂₀ heterocyclyl, and may be synthesised by reaction of a compound of formula 7 with a compound of formula R^{C1}COQ, in which R^{C3} is as previously defined and Q is a suitable leaving group, for example a halogen such as chloro, optionally in the presence of a base, for example pyridine, triethylamine or diisopropylethylamine, optionally in the presence of a solvent, for example dichloromethane, at a temperature in the range of 0° C. to the boiling point of the solvent used.

Compounds of formula 9 may also be synthesised by reaction of a compound of formula 7 with a compound of formula R^{C1}CO₂H, in which R^{C1} is as previously defined, in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine, in a solvent, for example dimethylacetamide or dichloromethane, at a temperature in the range of 0° C. to the boiling point of the solvent used.

Compounds of formula 1a in which R^{II} is an amido or thioamido moiety, can be represented by formula 10:

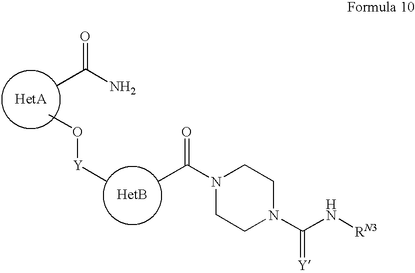

in which Y¹ is O or S, and R^{N3} is selected from the group consisting of optionally substituted C₁₋₂₀ alkyl, C₅₋₂₀ aryl and C₃₋₂₀ heterocyclyl, and may be synthesised by reaction of a compound of formula 7 with a compound of formula R^{N3}NC(═Y′), in which R^{N3} is as previously defined, in the presence of a solvent, for example dichloromethane, at a temperature in the range of 0° C. to the boiling point of the solvent used.

Compounds of formula 1a in which R^{II} is a sulfonyl moiety, can be represented by formula 11:

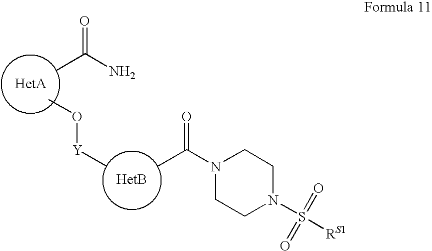

in which R^{S1} is selected from the group consisting of optionally substituted C₁₋₂₀ alkyl, C₅₋₂₀ aryl and C₃₋₂₀ heterocyclyl, and can be synthesised by reaction of a compound of formula 7 with a compound of formula R^{S1}SO₂Cl, in which R^{S1} is as previously defined, optionally in the presence of a base, for example pyridine, triethylamine or diisopropylethylamine, in the presence of a solvent, for example dichloromethane, at a temperature in the range of 0° C. to the boiling point of the solvent used.

Compounds of formula 8:

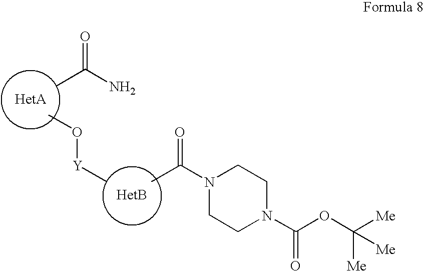

may also be synthesized from compounds of formula 12a:

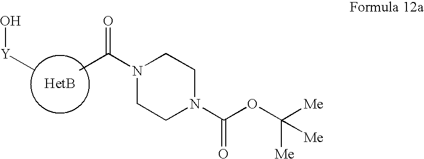

by Mitsunobu coupling with a compound of formula 5:

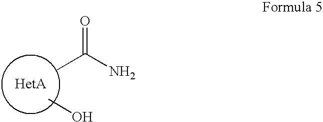

Compounds of formula 12a may be derived from compounds of formula 14:

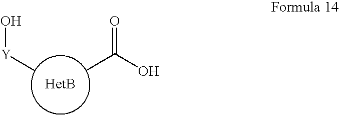

in an analgous way to compounds of formula 8 from compounds of formula 2.

Compounds of formula 8 may also be synthesized by joining a compound of formula 12b:

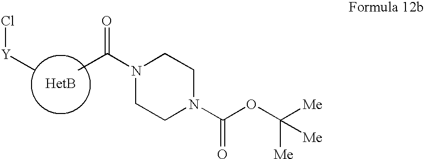

to a compound of formula 5:

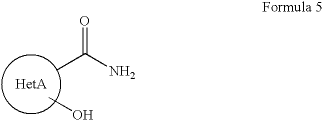

This coupling may be achieved by Mitsunobu reaction using a coupling reagent such as diisopropyl azodicarboxylate and triphenylphosphine in acetone.

Compounds of formula 12b can be derived from compounds of formula 12a by chlorination of the alcohol with reagents such as thionyl chlororide in choloroform at, for example, room temperature.

Compounds of formula 8 may also be synthesized by joining a compound of formula 12c:

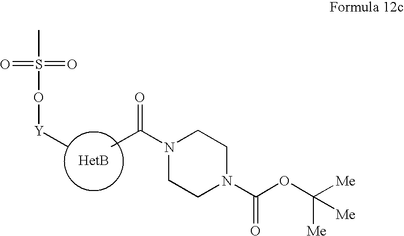

to a compound of formula 5:

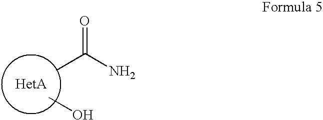

This coupling may be achieved by a Williamson ether formation between the alcohol and the mesylate.

Compounds of formula 12c can be derived from compounds of formula 12a by acylation with methane sulfonyl chloride in the presence of a suitable base.

### Use

The present invention provides active compounds, specifically, active in inhibiting the activity of PARP.

The term “active” as used herein, pertains to compounds which are capable of inhibiting PARP activity, and specifically includes both compounds with intrinsic activity (drugs) as well as prodrugs of such compounds, which prodrugs may themselves exhibit little or no intrinsic activity.

One assay which may conveniently be used in order to assess the PARP inhibition offered by a particular compound is described in the examples below.

The present invention further provides a method of inhibiting the activity of PARP in a cell, comprising contacting said cell with an effective amount of an active compound, preferably in the form of a pharmaceutically acceptable composition. Such a method may be practised in vitro or in vivo.

For example, a sample of cells may be grown in vitro and an active compound brought into contact with said cells, and the effect of the compound on those cells observed. As examples of “effect”, the amount of DNA repair effected in a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of active compounds in conjunction with known therapeutic means. Such means include cytotoxic regimes of drugs and/or ionising radiation as used in the treatment of different cancer types. In particular, the active compounds are known to potentiate the actions of a number of cancer chemotherapy treatments, which include the topoisomerase class of poisons (e.g. topotecan, irinotecan, rubitecan), most of the known alkylating agents (e.g. DTIC, temozolamide) and platinum based drugs (e.g. carboplatin, cisplatin) used in treating cancer.

Active compounds may also be used as cell culture additives to inhibit PARP, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti erbB2 antibody trastuzumab [HerceptinT], the anti-EGFR antibody panitumumab, the anti erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD 1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti vascular endothelial cell growth factor antibody bevacizumab (AvastinT) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU 11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and
(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

### Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichloro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

### General Experimental Methods

### Preparative HPLC

Method A: A Waters ZQ LC-MS system No. LAA 246 operating in electrospray ionization mode was used, using a Jones Genesis C18 column (4 μm 50 mm×4.6 mm). Mobile phases A (0.1% formic acid in water) and B (0.1% formic acid in acetonitrile) were used in a gradient as follows—the flow rate was 2.0 ml/min.

| Time (mins) | % A | % B |
|---|---|---|
| 3 | 95 | 5 |
| 6 | 5 | 95 |
| 10 | 5 | 95 |
| 10.5 | 95 | 5 |
| 14 | 95 | 5 |

Method B: As above, but with gradient as follows:

| Time (mins) | % A | % B |
|---|---|---|
| 1 | 95 | 5 |
| 5 | 5 | 95 |
| 9 | 5 | 95 |
| 9.5 | 95 | 5 |
| 13 | 95 | 5 |

### Analytical HPLC

Analytical HPLC was carried out as for the preparative HPLC, but with a gradient as follows:

| Time (mins) | % A | % B |
|---|---|---|
| 2 | 95 | 5 |
| 3 | 5 | 95 |
| 6 | 5 | 95 |
| 6.5 | 95 | 5 |
| 9 | 95 | 5 |

### NMR

¹H NMR and ¹³C NMR were recorded using Bruker DPX 300 spectrometer at 300 MHz and 75 MHz respectively. Chemical shifts were reported in parts per million (ppm) on the δ scale relative to tetramethylsilane internal standard. Unless stated otherwise all samples were dissolved in DMSO-d₆.

### Example 1

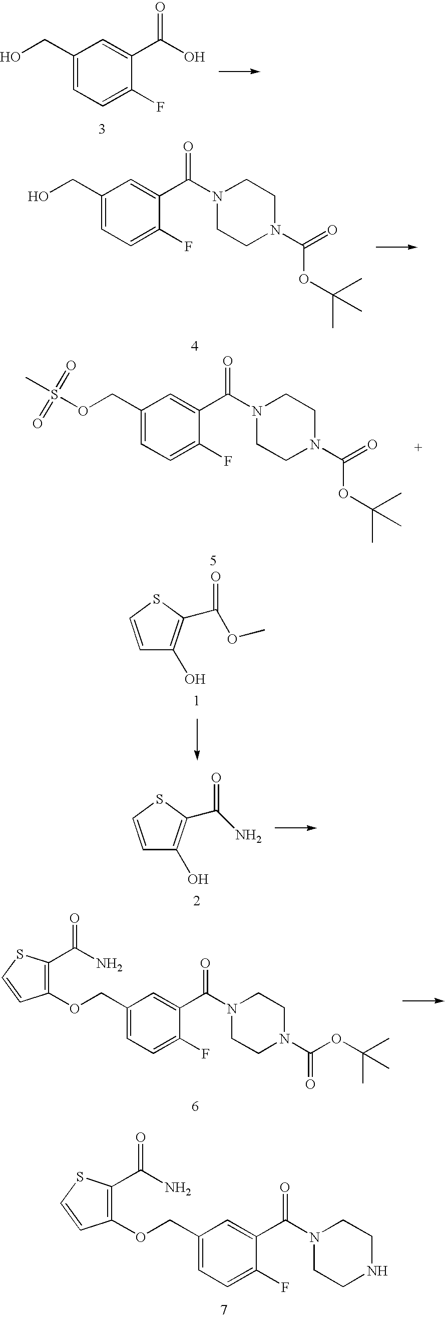

### (a) 3-Hydroxy-thiophene-2-carboxylic acid amide (2)

To a screw tight 150 ml pressure vessel was added 3-hydroxy-thiophene-2-carboxylic acid methyl ester (1)(5.0 g, 31.8 mmol) and ammonia in methanol (7N, 80 ml). The pressure vessel was sealed and contents stirred for 48 hours at 60° C. The reaction was cooled to room temperature and the solution evapourated to dryness to afford white crystalline solid (2). Single peak in LC-MS (4.9 g, 100% yield); m/z (LC-MS, ESP), RT=2.96 mins, (M+H) 144.

### (b) 4-(2-Fluoro-5-hydroxymethyl-benzoyl)-piperazine-1-carboxylic acid tert-butyl ester (4)

To 2-fluoro-5-hydroxymethyl-benzoic acid (3)(8.50 g, 50.0 mmol) in DMF (90 ml) at 20° C. was added triethylamine (13.8 ml, 100 mmol), followed by piperazine-1-carboxylic acid tert-butyl ester (11.16 g, 60.0 mmol) and HBTU (24.6 g, 65.0 mmol). A slight exotherm was noted and the reaction was stirred for 30 minutes. The reaction mixture was then cooled to 15° C., water (100 ml) added dropwise, forming an sticky yellow suspension. The aqueous liquor was extracted into DCM (3×80 ml), extracts washed with dilute sodium carbonate solution (2N, 100 ml), water (100 ml), dried over sodium sulfate, passed through a thin silica pad. Filtrate concentrated under vacuum to afford a colourless oil (4). Single peak in LC-MS (15.4 g, 91% yield) and taken through to next step without need for any further purification; m/z (LC-MS, ESP), RT=3.84 mins, (M+H) 339.

### (c) 4-(2-Fluoro-5-methanesulfonyloxymethyl-benzoyl)-piperazine-1-carboxylic acid tert-butyl ester (5)

To a solution of 4-(2-fluoro-5-hydroxymethyl-benzoyl)-piperazine-1 carboxylic acid tert-butyl ester (4)(6.8 g, 20.1 mmol) in dry DCM (60 ml) was added thriethyl amine (2.7 ml, 20.1 mmol). The resulting solution was cooled to 5° C., methane sulfonyl chloride (1.6 ml, 20.1 mmol) was added dropwise over 5 minutes. After 30 minutes the reaction was washed with water (2×50 ml) and dried over sodium sulfate to afford a tacky glass (5). Single peak in LC-MS (6.37 g, 76% yield) and taken through to next step without need for any purification; m/z (LC-MS, ESP), RT=4.20 mins, (M+H) 417.

### (d) 4-[5-(2-Carbamoyl-4-fluoro-phenoxymethyl)-2-fluoro-benzoyl]-piperazine-1-carboxylic acid tert-butyl ester (6)

To a solution of 4-(2-fluoro-5-methanesulfonyloxymethyl-benzoyl)-piperazine-1-carboxylic acid tert-butyl ester (5)(0.83 g, 2.0 mmol) in DMF (3 ml) under a nitrogen blanket was added 3-hydroxy-thiophene-2-carboxylic acid amide (2)(0.31 g, 2.1 mmol), followed by potassium carbonate (0.58 g, 4.2 mmol). The mixture was then heated to 90° C. for 30 minutes, water (20 ml) was added which resulted in a white precipitate. The aqueous phase was then extracted with DCM (3×20 ml) and dried over sodium sulfate, filtered and concentrated in vacuo. The crude oil was subjected to flash chromatography (eluent: neat DCM). A white solid (6) was isolated. Single peak in LC-MS (0.61 g, 63% yield) and taken through to next step without need for any further purification; m/z (LC-MS, ESP), RT=4.19 mins, (M+H) 474.

### (e) 3-[4-Fluoro-3-(piperazine-1-carbonyl)-benzyloxy]-thiophene-2-carboxylic acid amide (7)

To a solution of 4-[5-(2-carbamoyl-4-fluoro-phenoxymethyl)-2-fluoro-benzoyl]-piperazine-1-carboxylic acid tert-butyl ester (6)(0.45 g, 0.97 mmol) in dry DCM (3 ml) was added dropwise trifluoroacetic acid (3 ml) at room temperature. The reaction mixture was then concentrated in vacuo, resulting in a yellow oil (7). The crude reaction mixture was used directly in the next reaction and yield was taken to be quantitative. Single peak in LC-MS (quantitative yield) and taken through to next step without need for any purification; m/z (LC-MS, ESP), RT=3.15 mins, (M+H) 364.

### (g) Library Compounds

To a solution of 3-[4-fluoro-3-(piperazine-1-carbonyl)-benzyloxy]-thiophene-2-carboxylic acid amide (7)(0.069 mmol), Hunig's base (0.089 mmol) in DCM (0.5 ml), the appropriate acid chloride (0.089 mmol) was added. The reaction was stirred at room temperature for 16 hours and then concentrated in vacuo. The resulting materials were purified by preparative HPLC purification (Method A) to yield the compounds below:

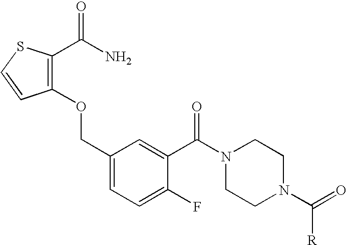

| Compound | R | Purity (%) | RT (min) | [M + H]⁺ |
|---|---|---|---|---|
| 8 | 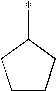 | 97 | 4.78 | 460.4 |
| 9 | 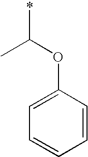 | 91 | 4.96 | 512.4 |
| 10 | 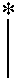 | 99 | 4.10 | 406.3 |
| 11 | 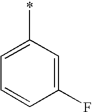 | 95 | 4.79 | 486.3 |
| 12 | 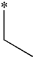 | 99 | 4.27 | 420.4 |
| 13 | 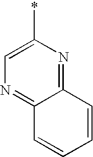 | 99 | 4.752 | 520.4 |
| 14 | 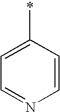 | 99 | 3.98 | 469.4 |

### Example 2

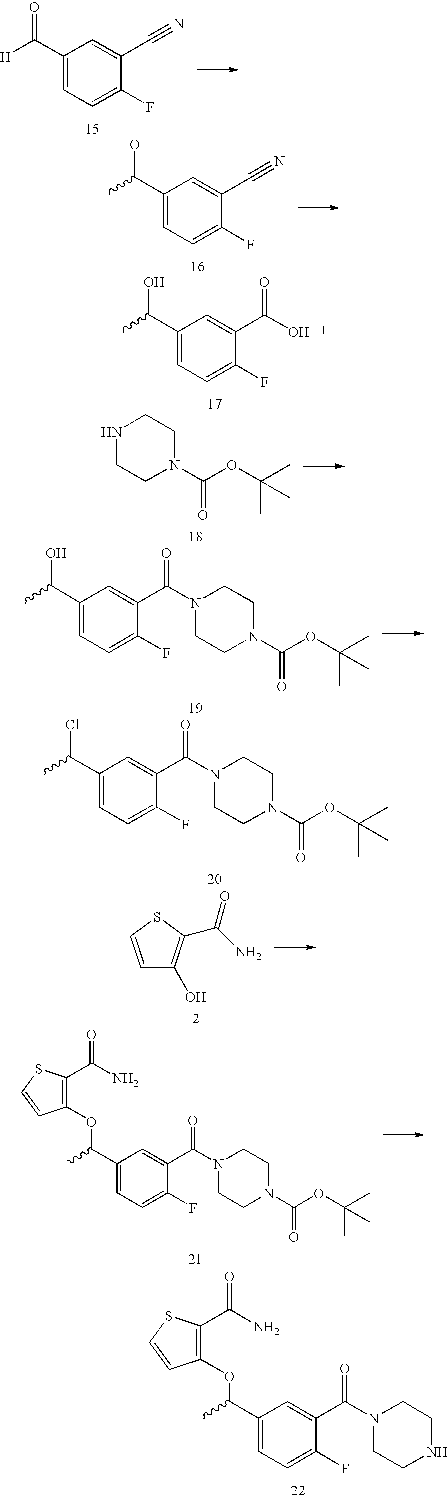

### (a) 2-Fluoro-5-(1-hydroxy-ethyl)-benzonitrile (16)

To a cooled solution of 2-fluoro-5-formyl-benzonitrile (15)(5.0 g, 33.6 mmol) in dry THF (100 ml) at 0° C. was added methyl magnesium bromide (33.9 mmol) dropwise. The reaction mixture was allowed to warm to room temperature over 10 minutes and then quenched by addition of saturated citric acid (10 ml). The reaction mixture was then concentrated in vacuo. The aqueous layer was then extracted with ethyl acetate (2×30 ml), dried over sodium sulfate, filtered and concentrated to afford a pale yellow oil.

The crude oil was subject to flash chromatography (eluent:hexane/ethyl acetate, 4:1, Rf 0.42 in ethyl acetate), a colourless oil (16) was isolated. Single peak in LC-MS analysis. (3.1 g, 56% yield) and required no further purification; m/z (LC-MS, ESP), RT=3.81 min, (M+H)=166.0.

### (b) 2-Fluoro-5-(1-hydroxy-ethyl)-benzoic acid (17)

To a solution of 2-fluoro-5-(1-hydroxy-ethyl)-benzonitrile (16)(1.8 g, 10.9 mmol) in water (20 ml) was added a solution of sodium hydroxide (12.2 g) dissolved in water (20 ml). The reaction mixture was heated at reflux for 40 minutes before being cooled to ambient temperature and diluted with water (40 ml). The mixture was then washed with ether (1×40 ml). The pH mixture was adjusted to (pH 2) with concentrated sulfuric acid a white solid precipitated resulted. The mixture was then extracted with ethyl acetate (5×40 ml). The ethyl acetate layers were then dried over magnesium sulfate, filtered and concentrated in vacuo to afford a pale yellow solid (17). Single peak in LC-MS analysis. (1.8 g, 89% yield) and required no further purification. m/z (LC-MS, ESN), RT=3.36 min, (M−H)=183.0.

### (c) 4-[2-Fluoro-5-(1-hydroxy-ethyl)-benzoyl]-piperazine-1-carboxylic acid tert-butyl ester (19)

To a solution of a 2-fluoro-5-hydroxymethyl-benzoic acid (17)(1.8 g, 9.8 mmol) in DCM (20 ml) was added O-benzotriazole-N,N,N′,N′-tetramethyl-uronium-hexafluoro-phosphate (4.2 g, 11.0 mmol), piperazine-1-carboxylic acid tert-butyl ester (18)(2.1 g, 11.0 mmol) and triethylamine (1.5 ml, 11.0 mmol). The mixture was and stirred at ambient temperature for 16 hours. The reaction mixture was then washed with saturated bicarbonate solution (2×20 ml). The organic layer dried over magnesium sulfate, filtered and concentrated in vacuo to a crude oil (5). The oil was then subjected to flash chromatography (eluent: hexane/ethyl acetate, 1:2, Rf 0.2). Single peak in LC-MS analysis. (1.8 g, 52% yield) and required no further purification. m/z (LC-MS, ESN), RT=4.09 mins, (M+H)=353.0.

### (d) 4-[5-(1-Chloro-ethyl)-2-fluoro-benzoyl]-piperazine-1-carboxylic acid tert-butyl ester (20)

To a solution of 4-[2-fluoro-5-(1-hydroxy-ethyl)-benzoyl]-piperazine-1-carboxylic acid tert-butyl ester (19)(1.8 g, 5.1 mmol) in DCM (18 ml) was added triethylamine (0.8 ml, 6.0 mmol). The reaction was cooled to 0° C. and mesyl chloride (1.3 ml, 17.2 mmol) and then pyridine (0.57 ml, 27.5 mmol). The reaction was then stirred overnight and then concentrated to dryness. Resulting in a 1:1 mixture of the chloride and mesylate adduct. The material was purified using silica gel (eluent: hexane/ethyl acetate, 4:1 (Rf 0.12). Polarity was gradually increased to neat ethyl acetate to remove the desired product (6) as a white solid. Single peak in LC-MS analysis. (370 mg, 20% yield); m/z (LC-MS, ESP), RT=4.73 mins, (M+1−t-butyl)=315.0.

### (e) 4-{5-[1-(2-Carbamoyl-thiophen-3-yloxy)-ethyl]-2-fluoro-benzoyl}-piperazine-1-carboxylic acid tert-butyl ester (21)

To a solution of 4-[5-(1-chloro-ethyl)-2-fluoro-benzoyl]-piperazine-1-carboxylic acid tert-butyl ester (20)(0.36 g, 0.84 mmol) in DMF (10 ml) was added 3-hydroxy-thiophene-2-carboxylic acid amide (2)(1.16 mmol) and potassium carbonate (0.32 g, 2.3 mmol). The mixture was then heated to 90° C. for 3 hours and then cooled to ambient temperature. The reaction was then diluted with water (15 ml) and extracted with ethyl acetate (2×10 ml). The combined organics were dried over magnesium sulfate, filtered and then concentrated in vacuo. The compound (21) was then submitted for preparative HPLC purification (Method B). Purity: 99%; Retention Time: 4.93 mins; M+H, 492.3.

### (f) 3-{1-[4-Fluoro-3-(piperazine-1-carbonyl)-phenyl]-ethoxy}-thiophene-2-carboxylic acid amide (22)

To a suspension of the appropriate 4-{5-[1-(2-carbamoyl-thiophen-3-yloxy)-ethyl]-2-fluoro-benzoyl}-piperazine-1-carboxylic acid tert-butyl ester (21)(0.117 mmol) in DCM (2 ml) was added trifluoroacetic acid (1.5 ml) and stirred for 1 hour at ambient temperature. The reaction mixtures was then concentrated in vacuo and the resulting compound submitted for preparative HPLC purification (Method B). Purity: 99%; Retention Time: 3.51 mins; M+H: 378.3.

### Example 3

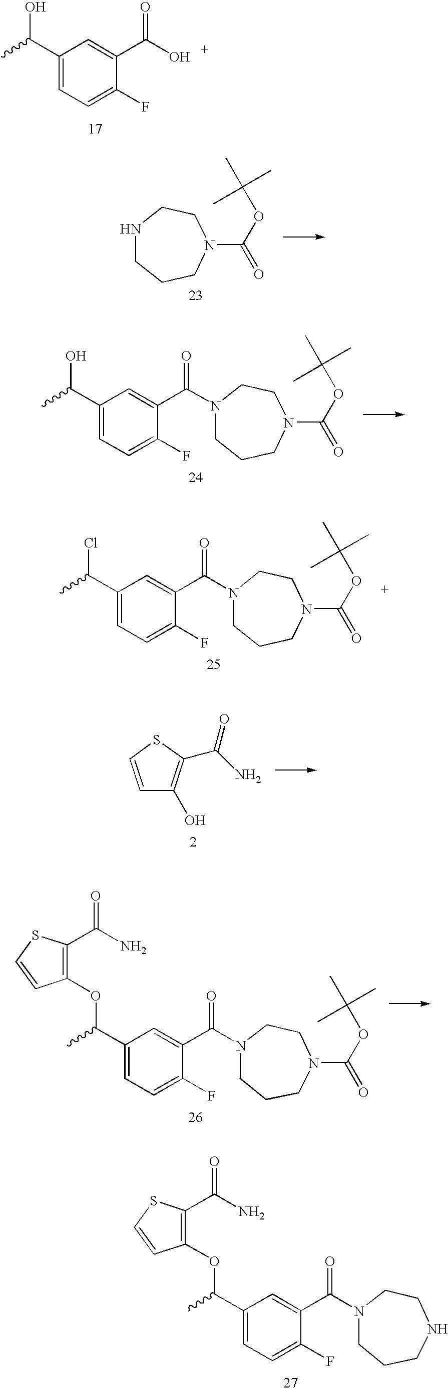

### (a) 4-[2-Fluoro-5-(1-hydroxy-ethyl)-benzoyl]-homo piperazine-1-carboxylic acid tert-butyl ester (24)

To a solution of a 2-fluoro-5-(1-hydroxy-ethyl)-benzoic acid (17)(4.9 g, 27.1 mmol) in DCM (20 ml) was added O-benzotriazole-N,N,N′,N′-tetramethyl-uronium-hexafluoro-phosphate (10.6 g, 28.00 mmol), homo-piperazine-1-carboxylic acid tert-butyl ester (23)(5.6 g, 28.0 mmol) and triethylamine (3.8 ml, 28.0 mmol). The mixture was and stirred at ambient temperature for 16 hours. The reaction mixture was then washed with saturated bicarbonate solution (2×20 ml). The organic layer dried over magnesium sulfate, filtered and concentrated in vacuo to a crude oil (24). The oil was then subjected to flash chromatography (eluent: neat ethyl acetate). Single peak in LC-MS analysis. (8.1 g, 82% yield) and required no further purification. m/z (LC-MS, ESN), RT=3.40 mins, (M+H)=367.0.

### (b) 4-[5-(1-Chloro-ethyl)-2-fluoro-benzoyl]-homo-piperazine-1-carboxylic acid tert-butyl ester (25)

To a solution of 4-[2-fluoro-5-(1-hydroxy-ethyl)-benzoyl]-homo-piperazine-1-carboxylic acid tert-butyl ester (24)(1.0 g, 2.7 mmol) in chloroform (30 ml) was added thionyl chloride (0.34 g, 2.71 mmol) and stirred for 30 minutes at room temperature. The reaction mixture was then concentrated to dryness to afford a beige solid (25). The material was taken through crude to next step without any purification.

### (c) 4-{5-[1-(2-Carbamoyl-thiophen-3-yloxy)-ethyl]-2-fluoro-benzoyl}-[1,4]diazepane-1-carboxylic acid tert-butyl ester (26)

To a solution of 4-[5-(1-chloro-ethyl)-2-fluoro-benzoyl]-homopiperazine-1-carboxylic acid tert-butyl ester (25)(0.17 g, 0.45 mmol) in DMF (0.5 ml) was added 3-hydroxy-thiophene-2-carboxylic acid amide (2)(0.45 mmol) and potassium carbonate (0.14 g, 0.9 mmol). The mixture was then heated to 90° C. for 3 hours and then cooled to ambient temperature. The compound (26) was then handed in for preparative HPLC for purification (Method B). Purity: 99%; Retention Time: 5.01 mins; M+H: 478.3.

### (d) 3-{1-[3-([1,4]Diazepane-1-carbonyl)-4-fluoro-phenyl]-ethoxy}-thiophene-2-carboxylic acid amide (27)

To a suspension of 4-{5-[1-(2-carbamoyl-thiophen-3-yloxy)-ethyl]-2-fluoro-benzoyl}-[1,4]diazepane-1-carboxylic acid tert-butyl ester (26)(0.2 mmol) in DCM (2 ml) was added trifluoroacetic acid (1.5 ml) and stirred for 1 hour at ambient temperature. The reaction mixture was then concentrated in vacuo and handed in for preparative HPLC purification (Method B). Purity: 99%; Retention Time: 3.55 mins; M+H: 392.3.

### Example 4

In order to assess the inhibitory action of the compounds, the following assay was used to determine IC₅₀ values or percentage inhibition at a given concentration.

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates (TRADE MARK) (NEN, UK) and varying concentrations of said inhibitors added. All compounds were diluted in DMSO and gave final assay concentrations of between 10 and 0.01 μM, with the DMSO being at a final concentration of 1% per well. The total assay volume per well was 40 μl.

After 10 minutes incubation at 30° C. the reactions were initiated by the addition of a 10 μl reaction mixture, containing NAD (5 μM), ³H-NAD and 30mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μl 30% acetic acid to each well. The plates were then shaken for 1 hour at room temperature.

The plates were transferred to a TopCount NXT (TRADE MARK) (Packard, UK) for scintillation counting. Values recorded are counts per minute (cpm) following a 30 second counting of each well.

The % enzyme activity for each compound is then calculated using the following equation:

$$\% Inhibition = 100 - ( 100 \times \frac{( \begin{matrix}cpm of unknowns - \\ mean negative cpm\end{matrix} )}{( \begin{matrix}mean positive cpm - \\ mean neagative cpm\end{matrix} )} )$$

IC₅₀ values (the concentration at which 50% of the enzyme activity is inhibited) were calculated, which are determined over a range of different concentrations, normally from 10 μM down to 0.001 μM. Such IC₅₀ values are used as comparative values to identify increased compound potencies.

The following compounds had an IC₅₀ of less than 2 μM: 6, 8-14, 21, 22, 26 and 27.

The Potentiation Factor (PF₅₀) for compounds is calculated as a ratio of the IC₅₀ of control cell growth divided by the IC₅₀ of cell growth+PARP inhibitor. Growth inhibition curves for both control and compound treated cells are in the presence of the alkylating agent methyl methanesulfonate (MMS). The test compounds were used at a fixed concentration of 0.5 micromolar. The concentrations of MMS were over a range from 0 to 10 μg/ml.

Cell growth was assessed using the sulforhodamine B (SRB) assay (Skehan, P., et al., (1990) New colorimetric cytotoxicity assay for anticancer-drug screening. J. Natl. Cancer Inst. 82, 1107-1112.). 2,000 HeLa cells were seeded into each well of a flat-bottomed 96-well microtiter plate in a volume of 100 μl and incubated for 6 hours at 37° C. Cells were either replaced with media alone or with media containing PARP inhibitor at a final concentration of 0.5, 1 or 5 μM. Cells were allowed to grow for a further 1 hour before the addition of MMS at a range of concentrations (typically 0, 1, 2, 3, 5, 7 and 10 μg/ml) to either untreated cells or PARP inhibitor treated cells. Cells treated with PARP inhibitor alone were used to assess the growth inhibition by the PARP inhibitor.

Cells were left for a further 16 hours before replacing the media and allowing the cells to grow for a further 72 hours at 37° C. The media was then removed and the cells fixed with 100 μl of ice cold 10% (w/v) trichloroacetic acid. The plates were incubated at 4° C. for 20 minutes and then washed four times with water. Each well of cells was then stained with 100 μl of 0.4% (w/v) SRB in 1% acetic acid for 20 minutes before washing four times with 1% acetic acid. Plates were then dried for 2 hours at room temperature. The dye from the stained cells was solubilized by the addition of 100 μl of 10 mM Tris Base into each well. Plates were gently shaken and left at room temperature for 30 minutes before measuring the optical density at 564 nM on a Microquant microtiter plate reader.

The following compounds had a PF₅₀ at 500 nM of at least 1.1: 9, 22 and 27.

1. A compound of the formula (I): 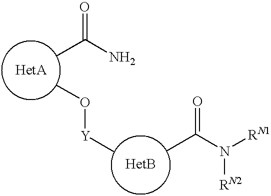
and pharmaceutically acceptable salts thereof, wherein:
HetA is a C₅ arylene group, wherein the two substituent groups are on adjacent ring atoms, and where the group is further optionally substituted by one halo, amino or C₁₋₇ alkoxy group;
Y is —CR^{C1}R^{C2}—(CH₂)ₘ—, where m is 0 or 1, R^{C1} is selected from H, CH₃ and CF₃, and R^{C2} is selected from H and CH₃, or R^{C1} and R^{C2} together with the carbon atom to which they are attached form the 1,1-cyclopropylene group: 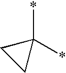
R^{N1} and R^{N2} are independently selected from H and R, where R is optionally substituted C₁₋₁₀ alkyl, C₃₋₂₀ heterocyclyl and C₅₋₂₀ aryl;
or R^{N1} and R^{N2}, together with the nitrogen atom to which they are attached form an optionally substituted 5-7 membered, nitrogen containing, heterocylic ring;
HetB is selected from:
(i) 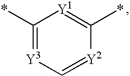
where Y¹ and Y³ are independently selected from CH and N, Y² is selected from CX and N and X is H, Cl or F; and
(ii) 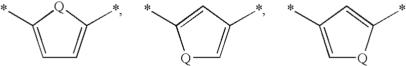
where Q is O or S.

2. A compound according to claim 1, wherein the optional substituent for HetA is selected from the group consisting of C₁₋₇ alkoxy, Cl and F.

3. A compound according to claim 1, wherein HetA is not substituted.

4. A compound according to claim 1, wherein HetA is a C₅ heteroarylene group containing a single ring heteroatom.

5. A compound according to claim 4, wherein HetA is derived from thiophene.

6. A compound according to claim 1, wherein m is 0.

7. A compound according to claim 1, wherein R^{C2} is H.

8. A compound according to claim 1, wherein R^{C1} is H.

9. A compound according to claim 1, wherein Het is:

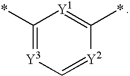

10. A compound according to claim 9, wherein up to two of Y¹, Y² and Y³ are N.

11. A compound according to claim 10, wherein one or none of Y¹, Y² and Y³ are N.

12. A compound according to claim 11, wherein either Y¹ or Y² is N.

13. A compound according to claim 9, wherein X is selected from H and F.

14. A compound according to claim 1, wherein Het is:

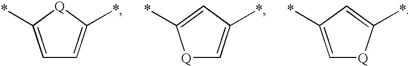

15. A compound according to claim 14, wherein Het is:

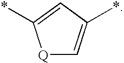

16. A compound according to claim 14, wherein Q is S.

17. A compound according to claim 1, wherein HetB is fluoro-phenylene, R^{C1} and R^{C2} are H and m is 0.

18. A compound according to claim 17, wherein HetA and its immediate substituents are:

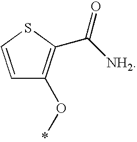

19. A compound according to claim 1, wherein R^{N1} is H and R^{N2} is R.

20. A compound according to claim 1, wherein R is optionally substituted C₁₋₇ alkyl or C₃₋₂₀ heterocylyl.

21. A compound according to claim 20, wherein R is a C₁₋₇ alkyl group which is unsubstituted or substituted with a single substituent, which is selected from a C₅₋₂₀ heterocyclic group, a C₅₋₂₀ aryl group, amino, halo, hydroxy, ether, thioether.

22. A compound according to claim 1, wherein R^{N1} and R^{N2}, together with the nitrogen atom to which they are attached form a group of formula II: 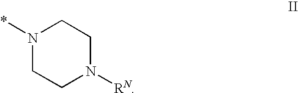
wherein R^{N} is selected from:
(i) —R^{II};
(ii) —C(═O)OR^{II}
(iii) —C(═O)NHR^{II};
(iv) —C(═S)NHR^{II};
(v) —S(═O)₂R^{II}; and
(vi) —C(═O)R^{II},
where R^{II} is selected from H, optionally substituted C₁₋₁₀ alkyl, C₃₋₂₀ heterocyclyl and C₅₋₂₀ aryl.

23. A compound according to claim 22, wherein R^{N} is selected from:
(i) —C(═O)NHR^{II};
(ii) —S(═O)₂R^{II}; and
(iii) —C(═O)R^{II}.

24. A compound according to claim 22, wherein R^{II} is selected from optionally substituted H, C₁₋₁₀ alkyl and C₅₋₂₀ aryl.

25. A compound according to claim 1, wherein R^{N1} and R^{N2}, together with the nitrogen atom to which they are attached form a group of formula III: 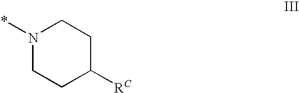
wherein R^{C} is preferably selected from the group consisting of: H; optionally substituted C₁₋₂₀ alkyl; optionally substituted C₅₋₂₀ aryl; optionally substituted C₃₋₂₀ heterocyclyl; optionally substituted acyl; optionally substituted amido; and optionally substituted ester groups.

26. A compound according to claim 25, wherein R^{C} is more selected from optionally substituted ester groups, wherein the ester substituent is a C₁₋₂₀ alkyl group.

27. A pharmaceutical composition comprising a compound according to claim 1, and a pharmaceutically acceptable carrier or diluent.

28. (canceled)

29. A method of inhibiting activity of PARP comprising contacting a cell with an effective amount of a compound according to claim 1.

30. (canceled)

31. A method of treating a condition comprising contacting a cell with an effective amount of a compound according to claim 1, wherein the condition is selected from vascular disease; septic shock; ischaemic injury, both cerebral and cardiovascular; reperfusion injury, both cerebral and cardiovascular; neurotoxicity; stroke; Parkinsons disease; haemorraghic shock; inflammatory diseases; arthritis, inflammatory bowel disease; ulcerative colitis; Crohn's disease; multiple sclerosis; secondary effects of diabetes; and cytoxicity following cardiovascular surgery.

32. A method of treating cancer comprising contacting a cell with ionizing radiation and an effective amount of a compound according to claim 1.

33. A method of treating cancer comprising contacting a cell with a chemotherapeutic agent and an effective amount of a compound according to claim 1.

34. A method of potentiating a tumor cell comprising contacting the tumor cell with an effective amount of a compound according to claim 1.

35. A method of treating cancer comprising contacting a cell with an effective amount of a compound according to claim 1, wherein the cancer is deficient in Homologous Recombination (HR) dependent DNA double strand break (DSB) repair activity.
